(19) United States
(12) Patent Application Publication
(10) Pub. No.: US 2009/0048619 A1
(43) Pub. Date: Feb. 19, 2009

(54) Capsulorhexis cutter and technique

(76) Inventor: Eitan Sobel, Osage Beach, MO (US)

Correspondence Address: Eitan Sobel, 2205 Portside LN, Osage Beach, MO, 65065, US

(21) Appl. No.: 11/893,491

(22) Filed: Aug. 17, 2007

(51) Int. Cl. A61F 9/007

(52) U.S. Cl. 606/166

(57) ABSTRACT

A device for performing a capsulorhexis incision inserted via a small wound cut in the corneal or sclera tissue. The device includes a motorized cutting element and a handle The motorized cutting element has mechanical means to create a cut in the anterior capsule tissue along a planned capsulorhexis line, said mechanical means tear small fragments of the anterior capsule in a fashion that may be similar to either a saw, a rotating chipping bit, a continuous hole puncher or a combination of those mechanical means thereby, creating a relatively smooth cut without the risk of undesired capsular tear.

U.S. Patent Documents

| | | | |
|---|---|---|---|
| 60/759,749 | January 2006 | Sobel, Eitan | A capsulorhexis device and technique. |
| 6,629,980 | October 2003 | Eibschitz-Tsimhoni; Maya | Instrument and method for creating an |
| | | | intraocular incision |
| 6,551,326 | April 2003 | Van Heugten; Anthony Y. | Capsulorhexis device |
| 6,306,155 | October 2001 | Chandler; Lamar | Capsulorhexis forceps and method of use |
| D437,413 | February 2001 | Chandler; Lamar | Capsulorhexis forceps with bellows |
| D437,054 | January 2001 | Chandler; Lamar | Capsulorhexis forceps |
| D436,663 | January 2001 | Chandler; Lamar | Capsulorhexis forceps with bulb |
| D436,662 | January 2001 | Chandler; Lamar | Capsulorhexis forceps with tab |
| 6,358,279 | March 2002 | Thai; Hessian | Minicapsulorhexis valve |
| 6,325,792 | December 2001 | Swinger; Casimir A. | Ophthalmic surgical laser and method |
| 5,873,883 | February 1999 | Cozean, Jr.; Charles H. | Hydraulic capsulorhexitome |
| 6,379,370 | April 2002 | Feinsod; Matthew | Incising apparatus for use in cataract |
| | | | surgery |
| 6,165,190 | December 2000 | Nguyen; Nhan | Capsulectomy device and method therefore |
| 5,669,923 | September 1997 | Gordon; Mark G. | Anterior capsulotomy device and procedure |
| 5,792,166 | August 1998 | Gordon; Mark G. | Anterior capsulotomy device and procedure |
| 5,728,117 | March 1998 | Lash; Roger S. | Retractable capsulorrehexis instument |
| 5,269,787 | December 1993 | Cozean, Jr.; Charles H. | Apparatus and method for capsulorhexis |
| D341,886 | November 1993 | Stolte; K. Broderick | Capsulorhexis forceps |
| 5,167,618 | December 1992 | Kershner; Robert M. | Capsulotomy forceps |
| 1/348157 | Aug 2006 | Sobel, Eitan | A capsulorhexis device and technique - |
| | | | Now Abandoned |

### BACKGROUND

1. Field of the Invention

The invention relates to surgical instruments for performing a circular opening in the anterior capsule covering the lens during cataract surgery. This procedure is called anterior capsulorectomy or capsulorhexis

2. Background of the Invention

The standard method of anterior capsulorectomy for phacoemulsification was developed by Gimbel and Neuhann. It begins by making a small incision in the anterior capsule at the center of the lens. The tissue is pulled towards the 12 o'clock position and a flap of tissue is thus created which is tearing in a circular fashion so that the tear force vector is tangential to the circumference of the tear circle. It is important to keep the grasping point of the flap close to the tear point. Sometimes, if the grasping point of the flap is too far, the tear may run “downhill” to the periphery and it may be extremely hard to redirect the tear back “uphill” to the desired circular pattern and may result in irreversible damage to the zonule.

Prior art (U.S. Pat. No. 6,551,326, Van Heugten; Anthony Y.) describes a device for making a circular cut comprising of a tubular outer member having a inner rod member made from an elastic spring material. When said inner rod member is slide out and extends beyond the open distal end of the tubular outer member, it forms a circular loop, a blade is attached to the distal tip of said circular loop and make a circular capsulorhexis.

Another prior are (U.S. Pat. No. 6,629,980, Eibschitz-Tsimhoni; Maya) Consisted of and instrument that includes a handle and a generally semicircular head element attached to the handle. This c shape is used to create a cut in the anterior chamber. However, it seems to us that having such a long cutting surface may not be an effective cutting tool and further tearing of the tissue would be required in order to complete the capsulorhexis.

Similar patents (U.S. Pat. No. 6,379,370, Feinsod; Matthew) and (U.S. Pat. No. 5,728,117, Lash; Roger S.) may create a complete round blade on top of the anterior chamber but again, the long cutting surface may fail to perform the required capsulorhexis. In addition, a whole ring pushed through the small incision in the cornea should be very flexible and potentially could tear the cornea or other structures in the anterior chamber. (U.S. Pat. No. 5,269,787; Cozean, Jr., et al.)

Other Methods used do not pertain directly to the current invention and include special forceps to hold the capsular flap (U.S. Pat. No. 5,167,618; Kershner and U.S. Pat. No. 6,306,155; Chandler, et al.) Another method consists of a special vacuum opening that is configured to draw in a element of a capsule when a vacuum is applied and forms a generally circular cut of a predetermined size (U.S. Pat. No. 5,669,923; Gordon)

### BRIEF SUMMARY OF THE INVENTION

The current invention is a device for performing a capsulorhexis incision. The device includes a handle attached to an elongated element having means at its distal end. Said elongated element is inserted via a small wound cut in the corneal or sclera tissue so that said cutting means may reach any part of the anterior capsule. The cutting means is designed to chip very small fragments of the anterior capsule, thereby creating a smooth cut in the anterior capsule. In this regard, the action of said cutting means is different than the current methods of cutting with a blade or alternatively, tearing the anterior capsule with a forceps or existing devices.

In its simplest form, said cutting means is a cylinder that moves up and down and as its moves down it punch a small hole in the anterior capsule tissue thereby creating a smooth cutting line.

Yet, a different design of said cutting means is a saw that by moving up and down, it is sawing the anterior capsule tissue. Since a saw is a flat strip, it may need to be rotated in the direction of the desired cut. Another important component of this cutting means design is the conduit end that touches the anterior capsule that may end with a flat plate which holds the tissue being cut in a fix position and facilitate the sawing action. A similar design could be based on a cutting cylinder that has several cutting rings on its distal end. Again, the conduit end that touches the anterior capsule or the end plate of the conduit can hold the tissue being cut in a fix position and even help the chipping process as tissue is trapped between the cutting rings and the conduit end that touches the anterior capsule or the end plate of the conduit.

A different method of a cutting means is a rotator cutter similar to a milling bit or a drill that as it rotates it cuts a smooth line.

The device is held by the handle that may contain mechanical parts that transmit motion to the cutting means via the elongated element. For example, the handle may contains power source, a motor or a vibrator and other mechanical parts.

In it simplest form, the operator moves the cutting means in a circular fashion. The operator may also follow a light marker on the anterior capsule that helps the operator to design the desired cut. A more complex methods to design a casulorhexis is to use a template as described in a prior art invention. In addition, the handle may contain a mechanism (mechanical or computerized) to move around the elongated element in a fashion that creates a circle within the anterior chamber helping the operator to created a round cut. A major advantaged of the invention is safety of the capsulorhexis process and preventing damage to the zonule. This enables the surgeon to make a larger capsulorhexis opening that prevents intra-operative and post operative capsular block.

The principals of the invention could be accomplished by different embodiments. The present disclosure is to be considered exemplary and it is not intended to limit the scope of the invention to these embodiments illustrated. The scope of the invention will be measured by the appended claims and their equivalents

### OBJECTS AND ADVANTAGES

Accordingly, several objects and advantages of the present invention are:
a) to provide a device that able to make a smooth cut in any direction the operator pleases, not being limited to a certain templates
b) to provide a mechanical method of cutting the anterior capsule without heat generation that can be created by a cauterizing device or a laser device.
c) to provide an operator the ability to create large and small capsulorhexis and to increase its size during the operation.

### BRIEF DESCRIPTION OF THE DRAWING

FIG. 1. The eye structure includes: the anterior capsule (1), the iris (2), the cornea (3). Although not part of this invention, the cutting line (4) of the capsulorhexis borders could be created by light template (not a part of this invention) or a real intra ocular template (not shown). The device has a handle (7) at its proximal end and an elongated conduit element (5) having a cutting means (6) at its distal end. The handle may contain a power source (8) to propel the cutting means. The cutting means is enlarged in circle (6). In this particular example, the conduit (5) has an inner cylinder (9) having cutting rings (11) that move up and down and are responsible to the cutting action of the device. (this cutting means also shown in FIG. 6)

FIG. 2. Simple cutting means based on a cylinder (9) moving up and down in a conduit (5) capable to punching holes in the anterior capsule at its distal end (10)

FIG. 3. Another cutting means having saw strip (11) capable moving up and down via a conduit (12). A mechanism (13) is available to rotate the saw strip (11) according to the direction of the desired cut.

FIG. 4. Yet, another example of cutting means having a cylinder (9) capable moving up and down via a conduit (5) having at its distal end a saw strip (11) capable of rotating in any direction.

FIG. 5. A different cutting means having a rotating cylinder (9) in a conduit (5) having at its distal a rotating cutter (12) similar to a milling bit or a drill creating a cut as it is moving horizontally.

FIG. 6. The cutting means shown in FIG. 1 wherein a cylinder (9) in a conduit (5) has at it distal end several cutting rings (11) that perform the cutting action by moving up and down

### REFERENCE NUMERALS IN DRAWING

1. the anterior capsule
2. the iris
3. the cornea
4. the cutting line
5. an elongated conduit element
6. the cutting means
7. a handle
8. a power source
9. an inner cylinder
10. hole puncher
11. a cutting saw or cutting wire with rings
12. saw strip conduit
13. a rotation mechanism
14. a rotating cutter

### DETAILED DESCRIPTION OF INVENTION

The present disclosure is to be considered as exemplary of the principles of the invention and it is not intended to limit the scope of the invention to these embodiments illustrated.

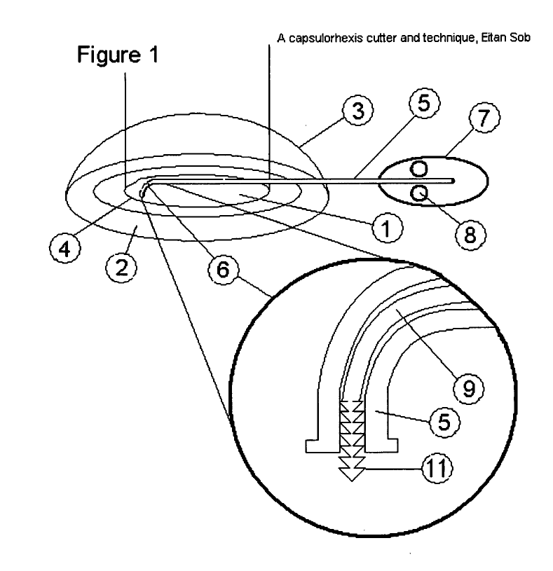

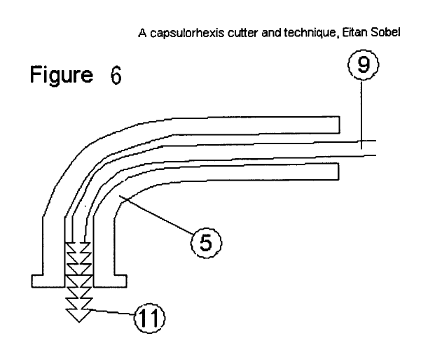

FIG. 1. The operator holds the device with its handle (7) and moving the cutting means (6) along the cutting line (4) on the anterior capsule tissue (1). This cutting line could be a light template or alternatively, a rigid intra ocular template could be used. The template is not an essential component and is used just for the convenience of the operator. The cutting element (6) in this example is shown in high power in circle (6) and later in FIG. 6.

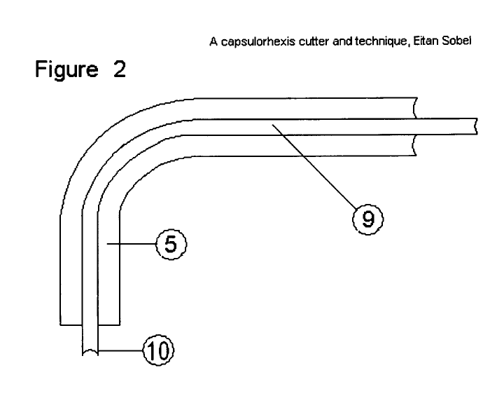

FIG. 2. This illustrates a hole puncher. Having the distal end (10) vibrating rapidly up and down and as the operator drag the cutting means may create the desired smooth cut along the cutting line.

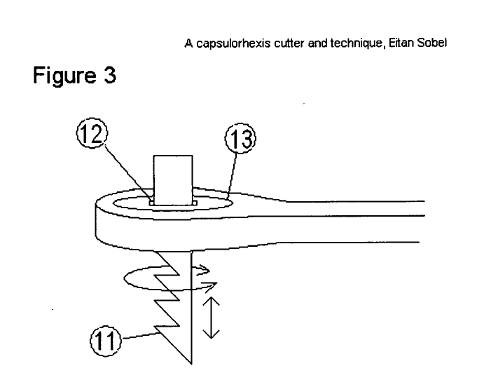

FIG. 3. A saw strip (11) capable vibrating up and down via a conduit (12) may be a more complicated method of cutting the anterior capsule tissue. The operator should rotate the narrow saw strip according to the direction of the desired cut.

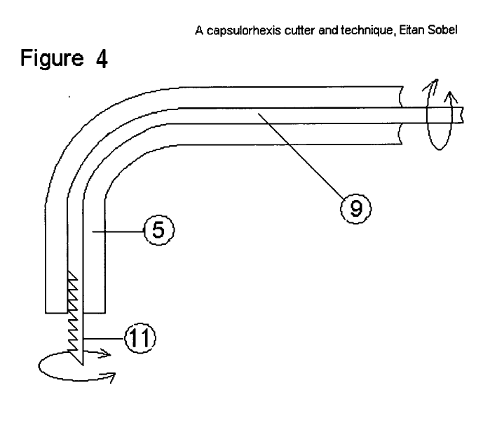

FIG. 4. Yet another example of a saw strip (11) pushed up and down by the internal cylinder (9) and making the desired cut. The operator may turn the saw strip (11) according to the direction of the desired cut. In this case, the combine effect of the saw strip moving against the base of a conduit (5) may facilitate the cutting action as tissue is being trapped between those elements.

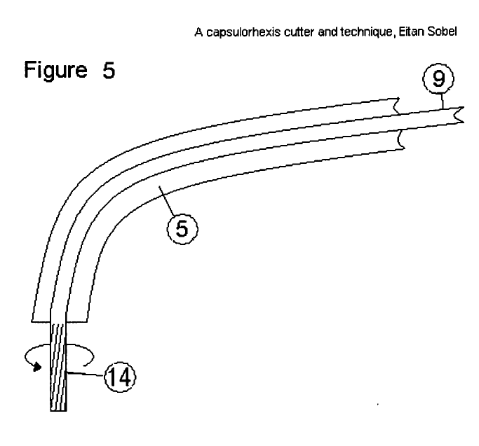

FIG. 5. The rotating cutter (14) is similar to the other cutting means, however, the rotating cutter (14) acts like a milling bit or a drill. The rotating of this cutter chips small tissue particles and thereby creates a smooth cutting line.

FIG. 6. The same operation as in FIG. 1 wherein the cutting mean consists of cutting rings (11) perform the cutting action by moving up and down and trapping small pieces of tissue between the rings (11) and the distal end of the conduit (5)

1. Device and technique for incising a thin sheet of tissues such as the anterior capsule of the eye, comprising of a handle, a shaft and a mechanical cutting means, wherein said cutting means is attached to the distal end of the shaft and is motorized by mechanical power transmitted from the handle via the shaft to said cutting element that either moves up and down and/or repeatedly rotates and therefore, is capable of mechanically sawing, cutting or chipping very small fragments of said thin sheet of tissues and as said cutting means is dragged on said thin sheet of tissues said cutting element is creating a relatively smooth cut, thereby, an operator holding the handle can insert said cutting element and distal portion of the shaft in small spaces such as anterior chamber of the eye, and said operator is able to drag said cutting means on said thin sheet of tissues to create a smooth cut in said thin sheet of tissue.

2. Said device and technique of claim 1 wherein said cutting means comprises of a toothed narrow band acting as a saw, said toothed narrow band moves repeatedly up and down or vibrates wherein part of said toothed narrow band uses a conduit in the distal end of said shaft and said toothed narrow band is penetrating and sawing said thin sheet of tissues of claim 1, furthermore, said toothed narrow band has a capability to turn to any direction, thereby, as said toothed narrow band can saw said thin sheet of tissues of claim 1 in any direction.

3. Said device and technique of claim 2 wherein the cutting is primarily done when said toothed narrow band of claim 2 is moving upward against said distal end of said conduit of claim 2 thereby the cutting action is done between said toothed narrow band and said distal end of said conduit of claim 2

4. Said device and technique of claim 1 wherein said cutting means comprises of a cylinder having a longitudinal notching similar to a drill or a milling bit, and said cylinder rotates around itself and is capable of chipping small fragment from said thin sheet of tissues, thereby, as said cutting means is dragged on said thin sheet of tissue, it creates a smooth cut in said thin sheet of tissue.

5. Said device and technique of claim 1 wherein said cutting means comprises of a cylinder having a bladed end or a knife at its distal end, said cylinder moves repeatedly up and down in a conduit element opened at its distal end, wherein said cylinder distal end can penetrate said thin sheet of tissue, thereby, behaving like a hole puncher or a can opener, furthermore, said distal end has a capability to turn to any direction, thereby, because of the repeatedly movements up and down of said distal end. it is able to create a smooth cut in said thin sheet of tissue.

6. Said device and technique of claim 1 wherein said cutting means comprises of a cylinder or a wire having spaced notches that function as cutter rings, the proximal part of said cylinder or wire with cutter rings moves repeatedly up and down in a conduit element opened at its distal end wherein said cylinder or wire with cutter rings can pass beyond said distal end and penetrate and saw said thin sheet of tissue in any direction without the need to rotate said cylinder or wire with cutter rings to the direction of said predetermined cutting line of claim 1.

7. Said device and technique of claim 6 wherein the cutting is primarily done when said separated cutter rings of claim 6 are moving upward against said distal end of said conduit of claim 6 thereby the cutting action is done between said cutter rings said distal end of said conduit of claim 6.

8. Said device and technique of claim 1 wherein said motorized mechanical cutting element is a combination of sawing action described in claim 2 or claim 6 and their dependent claims 3 and 7 respectively and the rotating drill like action of claim 4 or said hole punching or said can opening action of claim 5.

9. A device capable of cutting a superficial membrane tissue along a predetermined cutting line in close space such as the anterior chamber of the eye, comprising:
A. Sawing means moving repeatedly up and down vertically to said superficial membrane tissue and said sawing means is able to penetrate said superficial membrane tissue deep to the tissue beneath such as the lens of the eye, and capable of sawing said superficial membrane tissue at any direction desired.
B. an elongated conduit containing transmission means:
i. said transmission means is attached to said sawing means and transmit the power needed to move up and down said cutting means and, if needed, to rotate said cutting means to the desired direction.
ii. the distal end of said elongated conduit may assist the cutting action of said sawing means as fragments of said superficial membrane tissue may be trapped between said sawing means and said distal end of said elongated conduit;
C. a handle attached to the proximal end of said elongated conduit having motorized means necessary to be transmitted to said sawing means via said transmission means of said elongated conduit and said handle is used to hold and operate the device;
whereby an operator may hold the handle and insert said sawing means and the distal portion of said elongated conduit into a close space such as the anterior chamber of the eye and drag the said sawing means in any direction along said predetermined cutting line thereby creating a relatively smooth line cut in said superficial membrane tissue.

10. Said device and technique of claim 9 wherein said sawing means comprises of a toothed narrow band acting as a saw, said toothed narrow band moves repeatedly up and down and is penetrating said superficial membrane tissue of claim 9 into the deeper tissue laying beneath said superficial membrane tissue and said toothed narrow band is having a capability to turn to any direction, thereby said toothed narrow band can saw said superficial membrane of claim 9 in any direction said operator of claim 9 desires and create said predetermined cutting line of claim 9.

11. Said device and technique of claim 9 wherein said sawing means comprises of a sawing wire or sawing cylinder having spaced notches or cutter rings along and around said sawing wire or sawing cylinder, capable of sawing in any direction, wherein the movement of said sawing wire or sawing cylinder repeatedly up and down vertically to said superficial membrane tissue, said sawing wire or sawing cylinder can penetrate and saw said thin sheet of tissues of claim 9 saw in any direction desired by said operator of claim 9 without the need to rotate said sawing wire or sawing cylinder to the desired direction, thereby said sawing wire or sawing cylinder can saw said superficial membrane of claim 9 in any direction said operator of claim 9 desires and create said predetermined cutting line of claim 9.

12. A device capable of cutting a superficial membrane tissue along a predetermined cutting line in close space such as the anterior chamber of the eye, comprising:
A. a rotating cutting means compromising a rotating bit similar to a drill or to a milling bit said a rotating bit is capable of chipping small fragment from a superficial membrane tissue, thereby, creating a relatively smooth cut in said superficial membrane tissue in ant direction desired.
B. an elongated conduit containing transmission means:
i. said transmission means are attached to said rotating cutting means and transmit the power needed to rotate said rotating cutting means
ii. the distal end of said elongated conduit may assist the cutting action of said rotating cutting means as parts of said superficial membrane tissue may be trapped between said rotating cutting means and said distal end of said elongated conduit;
C. a handle attached to the proximal end of said elongated conduit having motorized means necessary to be transmitted to said rotating cutting means via said transmission means of said elongated conduit and said handle is used to hold and operate the device
whereby an operator may hold the handle and insert aid rotating cutting means and the distal portion of said elongated conduit into a close space such as the anterior chamber of the eye and drag the said rotating cutting means in any direction along a predetermined cutting line thereby creating a relatively smooth line cut in said superficial membrane tissue.

13. Said rotating cutting means of claim 12 wherein said rotating bit comprises of a cylinder having a longitudinal notching said cylinder rotates and is capable of chipping small fragment from said superficial membrane tissue. Of claim 12 thereby, said operator of claim 12 can move said rotating cutting means in any direction desired and creates a smooth cut along a predetermined cutting line.
